(19) United States
(12) Patent Application Publication
(10) Pub. No.: US 2026/0325003 A1
(43) Pub. Date: Oct. 22, 2026

(54) APPARATUS AND METHOD FOR MEASURING FAT OXIDATION BASED ON EXHALED BREATH USING MULTIPLE SENSORS

(71) Applicant: Electronics and Telecommunications Research Institute, Daejeon (KR)

(72) Inventors: Hyowoong NOH, Daejeon (KR); Dae-Sik LEE, Daejeon (KR); Byeongju LEE, Daejeon (KR); Junyeong LEE, Daejeon (KR); Hyung-Kun LEE, Daejeon (KR); Hyung Ju PARK, Daejeon (KR); Myungjoon KWACK, Daejeon (KR); Doyeob KIM, Daejeon (KR)

(21) Appl. No.: 19/568,628

(22) Filed: Mar. 16, 2026

(30) Foreign Application Priority Data
Apr. 17, 2025 (KR) 10-2025-0050457

(51) Int. Cl. A61B 5/08; A61B 5/00; A61B 5/097

(52) U.S. Cl. CPC A61B 5/082; A61B 5/097; A61B 5/7225; A61B 5/7271; A61B 2562/0285; A61B 2562/06

(57) ABSTRACT

An apparatus for measuring fat oxidation is provided. The apparatus comprises a sample injection unit into which exhaled breath of a user is introduced, a breath measurement unit configured to detect metabolites in the exhaled breath, a signal analysis unit configured to analyze the detected metabolites, a flow control unit configured to automatically control a flow rate of the exhaled breath, and an exhaust unit configured to discharge the exhaled breath to the outside.

### CROSS-REFERENCE TO RELATED APPLICATIONS

This application claims priority under 35 U.S.C. § 119 to Korean Patent Application No. 10-2025-0050457 filed on Apr. 17, 2025, in the Korean Intellectual Property Office, the disclosures of which are incorporated by reference herein in their entireties.

### BACKGROUND

The present disclosure relates to a fat oxidation measurement apparatus based on exhaled breath, and more specifically, to a fat oxidation measurement apparatus that measures and analyzes metabolites in breath using multiple sensors and operating method thereof.

According to the Korea National Health and Nutrition Examination Survey by the Korea Disease Control and Prevention Agency, the adult obesity rate in Korea (the proportion of adults having a body mass index (BMI) of 25 or more) has increased by about 20.4% compared to that in 2014(30.9 %). Obesity is broadly classified into primary obesity (obesity caused by an increase in body fat due to energy intake exceeding energy expenditure) and secondary obesity (obesity caused by neurological and endocrine diseases, genetic and congenital disorders, etc.), and primary obesity accounts for 90% or more of all obesity cases. Since methods for overcoming primary obesity vary depending on individual physical and environmental conditions, demand is increasing for systems capable of systematically and accurately measuring a body fat oxidation state for more efficient dieting.

Currently, a most widely used method is a bio-impedance measurement method in which a micro-current is applied to a human body and impedance differences between muscle having a high water content and fat having a low water content are measured to estimate body water, protein, minerals, and fat. Although this method is non-invasive and provides information by body part, it has limitations in that accuracy is not high, the method is relatively expensive, portability is low, and it cannot be used by patients with implanted electronic devices. In addition, although methods using mobile devices have recently been used, most of them merely measure heart rate or the number of steps to simply calculate calorie consumption, and thus there is an increasing need for more accurate and systematic measurement devices.

### SUMMARY

An object of the present invention is to provide an apparatus for measuring fat oxidation and an operating method thereof, capable of measuring multiple metabolites in a user's oral breath gas and alveolar breath gas and analyzing a progress of fat oxidation in the body based thereon, thereby ultimately monitoring a user's dieting state in real time and maximizing dieting efficiency.

According to one embodiment of the present disclosure, an apparatus for measuring fat oxidation includes a sample injection unit into which a user's exhaled breath is introduced, a breath measurement unit configured to detect metabolites in the exhaled breath, a signal analysis unit configured to analyze the detected metabolites, a flow control unit configured to automatically control a flow rate of the exhaled breath, and an exhaust unit configured to discharge the exhaled breath to the outside.

In one embodiment, the sample injection unit comprises a straw configured to directly contact an oral cavity of the user, a differential pressure sensor configured to detect introduction of exhaled breath into the breath measurement unit, a flow sensor configured to control a volume of exhaled breath introduced into the breath measurement unit, and a carbon dioxide sensor configured to classify the exhaled breath into an oral breath gas and an alveolar breath gas.

In one embodiment, the straw further comprises a droplet filter configured to block droplets.

In one embodiment, the breath measurement unit comprises a first gas chamber configured to store oral breath gas included in the exhaled breath, and a second gas chamber configured to store alveolar breath gas included in the exhaled breath.

In one embodiment, the first gas chamber comprises a first gas sensor array configured to detect metabolites in the exhaled breath, and the second gas chamber includes a second gas sensor array configured to detect metabolites in the exhaled breath.

In one embodiment, the first gas sensor array comprises at least one of a semiconductor gas sensor, an electrochemical gas sensor, a photoionization gas sensor, a metal organic framework (MOF) gas sensor, a carbon nanowall (CNW) gas sensor, and a bioreceptor gas sensor, and the second gas sensor array comprises the same gas sensors as the first gas sensor array.

In one embodiment, the signal analysis unit comprises a preprocessing unit configured to preprocess metabolite data signals detected by the breath measurement unit, and a calculation unit configured to compute and analyze a concentration and composition of metabolites included in the exhaled breath based on the preprocessed signals.

In one embodiment, the preprocessing unit comprises an instrumentation amplifier circuit configured to maximize a signal-to-noise ratio (SNR) of metabolite data signals measured by the breath measurement unit, an automatic amplification circuit configured to remove an offset of the measured signals, and a capacitive filter configured to remove high-frequency noise and white noise of the measured signals.

In one embodiment, the calculation unit comprises a computing circuit configured to normalize data based on the preprocessed signals and generate two-dimensional response map data, and an evaluation circuit configured to analyze results of the computing circuit.

In one embodiment, the flow control unit comprises a first flow control unit connected to the sample injection unit, a second flow control unit connected between the sample injection unit and the breath measurement unit, and a third flow control unit connected between the breath measurement unit and the exhaust unit.

In one embodiment, the first flow control unit comprises a first fluid control valve, the second flow control unit includes a second fluid control valve and a first three-way valve, and the third flow control unit includes a third fluid control valve and a second three-way valve.

In one embodiment, the exhaust unit further comprises an exhaust pump configured to discharge residual exhaled breath to the outside.

According to one embodiment of the present disclosure, an operating method of an apparatus for measuring fat oxidation, for measuring metabolites in exhaled breath and measuring a fat oxidation state in a body, comprises introducing exhaled breath of a user into the apparatus by a sample injection unit included in the apparatus, detecting metabolites included in the exhaled breath by a breath measurement unit included in the apparatus, analyzing the metabolites by a signal analysis unit included in the apparatus, and discharging the exhaled breath to the outside by an exhaust unit included in the apparatus.

In one embodiment, the method further comprises controlling the flow rate of the exhaled breath in real time by a flow control unit included in the apparatus.

In one embodiment, introducing the exhaled breath into the apparatus comprises measuring a pressure difference of the exhaled breath and a carbon dioxide ratio of the exhaled breath by a differential pressure sensor and a carbon dioxide sensor included in the sample injection unit, opening a second fluid control valve and a third fluid control valve by the flow control unit included in the apparatus, blocking a second port of each of a first three-way valve and a second three-way valve by the flow control unit, introducing the exhaled breath into a first gas chamber of the breath measurement unit by the flow control unit, and operating an exhaust pump included in the exhaust unit by the exhaust unit.

In one embodiment, introducing the exhaled breath of the user into the apparatus further comprises measuring a volume of exhaled breath within the first gas chamber by a flow sensor included in the sample injection unit, measuring a carbon dioxide ratio of the exhaled breath by the carbon dioxide sensor included in the sample injection unit, blocking a first port of each of the first three-way valve and the second three-way valve by the flow control unit, opening a second port of the first three-way valve and the second three-way valve by the flow control unit, introducing the exhaled breath into a second gas chamber of the breath measurement unit by the flow control unit, and operating the exhaust pump included in the exhaust unit by the exhaust unit.

In one embodiment, measuring metabolites included in the exhaled breath comprises measuring volumes of exhaled breath introduced into the first gas chamber and the second gas chamber of the breath measurement unit by the flow sensor, opening a first fluid control valve and blocking a second fluid control valve and a third fluid control valve by the flow control unit, blocking second ports of the first three-way valve and the second three-way valve by the flow control unit, and measuring metabolites included in the exhaled breath by gas sensor arrays included in the breath measurement unit.

In one embodiment, analyzing the metabolites comprises preprocessing metabolite data by a preprocessing unit included in the signal analysis unit, normalizing the preprocessed data and generating two-dimensional response map data based on the normalized data by a calculation unit included in the signal analysis unit, and analyzing a concentration and composition of metabolites included in the exhaled breath by the calculation unit included in the signal analysis unit.

In one embodiment, discharging the exhaled breath to the outside comprises opening a first port and a second port of a first three-way valve by the flow control unit, opening a first port and a second port of a second three-way valve by the flow control unit, opening a second fluid control valve and a third fluid control valve by the flow control unit, and operating an exhaust pump included in the exhaust unit by the exhaust unit.

In one embodiment, discharging the exhaled breath to the outside further includes blocking first to third fluid control valves included in the apparatus by the flow control unit.

### BRIEF DESCRIPTION OF THE FIGURES

The above and other objects and features of the present disclosure will become apparent by describing in detail embodiments thereof with reference to the accompanying drawings.

FIG. 1 illustrates a configuration of an apparatus for measuring fat oxidation according to an embodiment of the present disclosure.

FIG. 2 illustrates a detailed configuration of the apparatus for measuring fat oxidation shown in FIG. 1.

FIG. 3 illustrates components included in a signal analysis unit of the apparatus for measuring fat oxidation.

FIG. 4 is a flowchart illustrating an operating method of the apparatus for measuring fat oxidation shown in FIGS. 1 and 2.

FIG. 5 illustrates a state in which the apparatus for measuring fat oxidation is in a standby state.

FIG. 6 is a flowchart illustrating an operating method of the apparatus for measuring fat oxidation when oral breath gas is introduced.

FIG. 7 illustrates a flow of oral breath gas in the apparatus for measuring fat oxidation.

FIG. 8 is a flowchart illustrating an operating method of the apparatus for measuring fat oxidation when alveolar breath gas is introduced.

FIG. 9 illustrates a flow of alveolar breath gas in the apparatus for measuring fat oxidation.

FIG. 10 is a flowchart illustrating an operating method for analyzing metabolites in the apparatus for measuring fat oxidation.

FIG. 11 illustrates a flow of exhaled breath during metabolite analysis in the apparatus for measuring fat oxidation.

FIG. 12 is a flowchart illustrating an operating method for discharging exhaled breath in the apparatus for measuring fat oxidation.

FIG. 13 illustrates flows of external air introduced from outside and residual exhaled breath discharged to the outside in the apparatus for measuring fat oxidation.

FIG. 14 illustrates an example of a data normalization process and a two-dimensional data map generation process in a computing circuit.

FIG. 15 is a schematic diagram illustrating a process of analyzing metabolites in exhaled breath using an AI algorithm in an evaluation circuit.

FIG. 16 is a block diagram illustrating an electronic device according to embodiments of the present disclosure.

### DETAILED DESCRIPTION

Hereinafter, embodiments of the present disclosure will be described clearly and in detail such that a person having ordinary skill in the art to which the present disclosure pertains can readily practice the present disclosure.

Terms such as “unit” and “module” used herein, or functional blocks illustrated in the drawings, may be implemented in the form of software, hardware, or a combination thereof. In the following description, detailed descriptions of well-known components will be omitted to clearly explain the technical spirit of the present invention.

As used herein, expressions such as “A or B,” “at least one of A and B,” “at least one of A or B,” “A, B, or C,” “at least one of A, B, and C,” and “at least one of A, B, or C” may include any one of the listed items or any combination thereof.

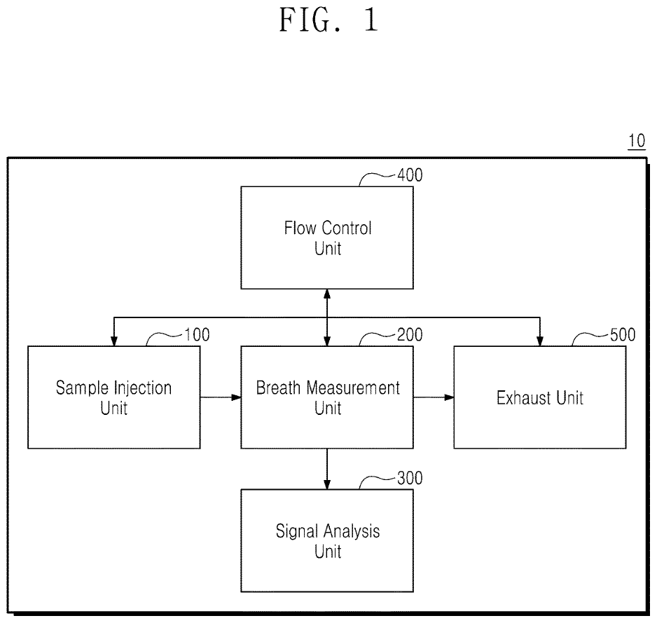

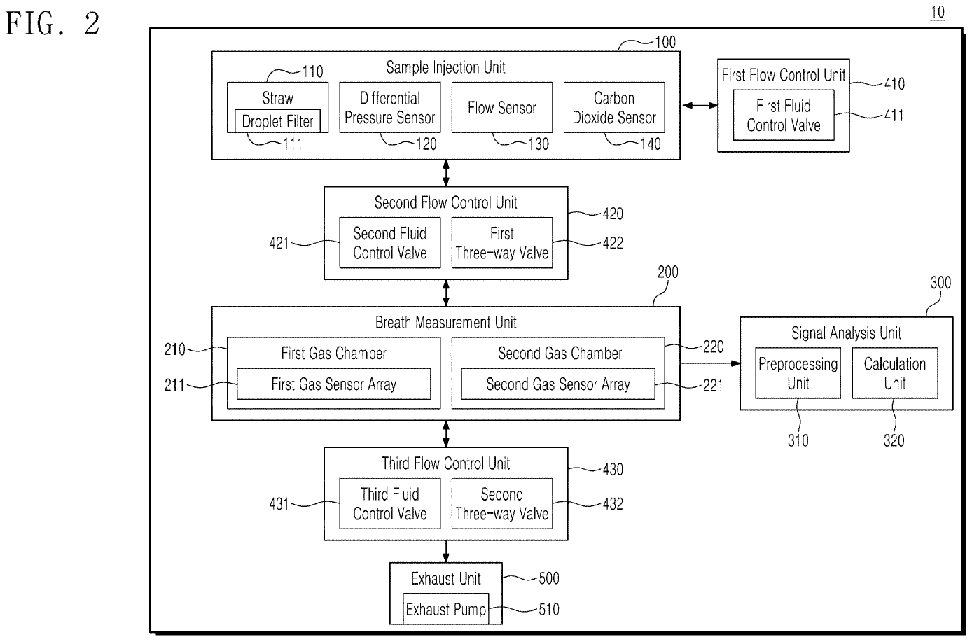

FIG. 1 illustrates a configuration of an apparatus for measuring fat oxidation 10 according to an embodiment of the present disclosure. FIG. 2 illustrates a detailed configuration of the apparatus for measuring fat oxidation 10 of FIG. 1.

Referring to FIGS. 1 and 2, the apparatus for measuring fat oxidation 10 may include a sample injection unit 100, a breath measurement unit 200, a signal analysis unit 300, a flow control unit 400, and an exhaust unit 500.

The sample injection unit 100 may include a straw 110, a differential pressure sensor 120, a flow sensor 130, and a carbon dioxide sensor 140. A user may directly introduce exhaled breath into the apparatus for measuring fat oxidation 10 through the straw 110. The differential pressure sensor 120 may detect exhaled breath introduced into the sample injection unit 100 through the straw 110. The flow sensor 130 may control a volume of exhaled breath introduced into the breath measurement unit 200. The carbon dioxide sensor 140 may classify the exhaled breath into oral breath gas (outer breath) and alveolar breath gas (inner breath). The sample injection unit 100 may include a gas pipe through which the exhaled breath flows.

In one embodiment, the straw 110 may be disposable. The straw 110 may be replaceable after use. The straw 110 may be in direct contact with a user's mouth.

In one embodiment, the straw 110 may include a droplet filter 111. The droplet filter 111 may block droplets that may be introduced during injection of exhaled breath by the user.

In one embodiment, the differential pressure sensor 120 may be disposed inside a gas pipe on a side of the sample injection unit 100. The carbon dioxide sensor 140 may be disposed inside the gas pipe on the side of the sample injection unit 100.

In one embodiment, the differential pressure sensor 120 may measure a pressure difference of introduced exhaled breath. When a pressure difference measured by the differential pressure sensor 120 is equal to or less than a preset value and a carbon dioxide ratio of the exhaled breath measured by the carbon dioxide sensor 140 corresponds to a preset ratio, the differential pressure sensor 120 and the carbon dioxide sensor 140 may transmit an opening signal to the flow control unit 400.

In one embodiment, the flow sensor 130 may be disposed inside the gas pipe on the side of the sample injection unit 100. The flow sensor 130 may measure a volume of exhaled breath introduced into the breath measurement unit 200. When the volume of exhaled breath introduced into the breath measurement unit 200 reaches a reference value, the flow sensor 130 may transmit a blocking signal to the flow control unit 400.

The breath measurement unit 200 may include a first gas chamber 210 and a second gas chamber 220. The first gas chamber 210 may accommodate oral breath gas included in the exhaled breath and provide an environment for analyzing components of the oral breath gas. The second gas chamber 220 may accommodate alveolar breath gas included in the exhaled breath and provide an environment for analyzing components of the alveolar breath gas. Types of gas accommodated in the first gas chamber 210 and the second gas chamber 220 may be changed. The breath measurement unit 200 may include a gas pipe through which the exhaled breath flows.

In one embodiment, the first gas chamber 210 may include a first gas sensor array 211. The second gas chamber 220 may include a second gas sensor array 221. The first gas sensor array 211 and the second gas sensor array 221 may include one or more of a semiconductor gas sensor, an electrochemical gas sensor, a photoionization gas sensor, a metal organic framework (MOF) gas sensor, a carbon nanowall (CNW) gas sensor, and a bioreceptor gas sensor. Configurations of gas sensors constituting the first gas sensor array 211 and the second gas sensor array 221 may be identical.

In one embodiment, the first gas sensor array 211 and the second gas sensor array 221 may simultaneously detect various metabolites. The metabolites may include ketone bodies such as acetone, acetoacetate, and β-hydroxybutyrate (βBOHB), free fatty acids (FFA), glucose, and the like.

In one embodiment, the first gas chamber 210 and the second gas chamber 220 may be designed based on results of fluid simulation. The first gas chamber 210 and the second gas chamber 220 may maximize detection sensitivity of metabolites.

The signal analysis unit 300 may include a preprocessing unit 310 and a calculation unit 320. The preprocessing unit 310 may preprocess metabolite data detected by the breath measurement unit 200. The calculation unit 320 may analyze metabolites based on the preprocessed data.

The flow control unit 400 may include first to third flow control units 410, 420, and 430. The first flow control unit 410 may include a first fluid control valve 411. The second flow control unit 420 may include a second fluid control valve 421 and a first three-way valve 422. The third flow control unit 430 may include a third fluid control valve 431 and a second three-way valve 432.

In one embodiment, the first fluid control valve 411 may be connected to a portion of the sample injection unit 100 to which the straw 110 is not connected. The second fluid control valve 421 may be connected between a gas pipe of the sample injection unit 100 and a gas pipe of the breath measurement unit 200. The third fluid control valve 431 may be connected between the gas pipe of the breath measurement unit 200 and a gas pipe of the exhaust unit 500.

In one embodiment, when the blocking signal is transmitted from the flow sensor 130 of the sample injection unit 100, the flow control unit 400 may open the first fluid control valve 411. When the blocking signal is transmitted from the flow sensor 130 of the sample injection unit 100, the flow control unit 400 may block the second fluid control valve 421 and the third fluid control valve 431.

In one embodiment, when the opening signal is transmitted from the differential pressure sensor 120 of the sample injection unit 100, the flow control unit 400 may block the first fluid control valve 411. When the opening signal is transmitted from the differential pressure sensor 120 of the sample injection unit 100, the flow control unit 400 may open the second fluid control valve 421 and the third fluid control valve 431.

In one embodiment, when a discharge start signal is transmitted from the breath measurement unit 200, the flow control unit 400 may open the first to third fluid control valves 411, 421, and 431.

In one embodiment, the first three-way valve 422 may be positioned in a gas pipe connecting the sample injection unit 100 and the breath measurement unit 200. The second three-way valve 432 may be positioned in a gas pipe connecting the breath measurement unit 200 and the exhaust unit 500.

In one embodiment, the first three-way valve 422 and the second three-way valve 432 may selectively send the exhaled breath to the first gas chamber 210 or the second gas chamber 220.

The exhaust unit 500 may include an exhaust pump 510 and a discharge outlet. The exhaust pump 510 may discharge remaining exhaled breath in the apparatus for measuring fat oxidation 10 to the outside through the discharge outlet. The exhaust unit 500 may include a gas pipe through which the exhaled breath flows.

In one embodiment, the exhaust unit 500 may control a speed of the exhaust pump 510 based on a volume of exhaled breath introduced into the breath measurement unit 200. When the second fluid control valve 421 and the third fluid control valve 431 are blocked, the exhaust unit 500 may stop operation of the exhaust pump 510. When the second fluid control valve 421 and the third fluid control valve 431 are opened, the exhaust pump 510 may operate at a high speed. When exhaled breath is introduced into the first gas chamber 210 and the second gas chamber 220, the exhaust pump 510 may operate at a low speed.

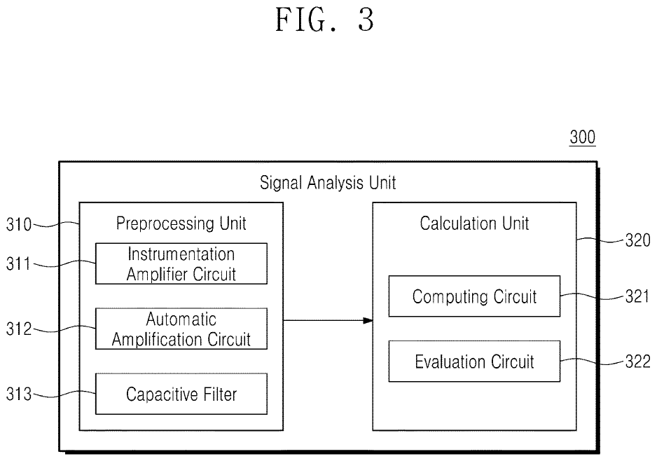

FIG. 3 illustrates components included in the signal analysis unit 300 of the apparatus for measuring fat oxidation 10. Referring to FIG. 3, the preprocessing unit 310 of the signal analysis unit 300 may include an instrumentation amplifier circuit 311, an automatic amplification circuit 312, and a capacitive filter 313. The calculation unit 320 of the signal analysis unit 300 may include a computing circuit 321 and an evaluation circuit 322.

The instrumentation amplifier circuit 311 may be configured to maximize a signal-to-noise ratio (SNR) of data signals detected by the first gas sensor array 211 and the second gas sensor array 221. The automatic amplification circuit 312 may be configured to remove(cancel) a DC offset included in data signals detected by the first gas sensor array 211 and the second gas sensor array 221. The capacitive filter 313 may be configured to remove(cancel) high-frequency noise and white noise of data signals detected by the first gas sensor array 211 and the second gas sensor array 221.

In one embodiment, the preprocessing unit 310 may be configured such that the instrumentation amplifier circuit 311, the automatic amplification circuit 312, and the capacitive filter 313 operate simultaneously. The instrumentation amplifier circuit 311, the automatic amplification circuit 312, and the capacitive filter 313 may include independent circuit blocks or digital algorithms. The instrumentation amplifier circuit 311, the automatic amplification circuit 312, and the capacitive filter 313 may operate in parallel with respect to a signal.

The computing circuit 321 may normalize data preprocessed by the preprocessing unit 310. The computing circuit 321 may generate two-dimensional response map data based on the normalized data. The evaluation circuit 322 may analyze metabolites in exhaled breath based on the two-dimensional response map data.

In one embodiment, the computing circuit 321 may generate two-dimensional response map (2D response map) data having a size of [number of gas sensors×measurement time].

In one embodiment, the evaluation circuit 322 may operate based on an AI (Artificial Intelligence) algorithm. The evaluation circuit 322 may use a learning algorithm based on a one-dimensional convolutional neural network (1D-CNN) suitable for analyzing normalized time-series data. The evaluation circuit 322 may extract feature points while learning the normalized data. The evaluation circuit 322 may analyze a concentration and composition of metabolites in exhaled breath based on the two-dimensional response map data.

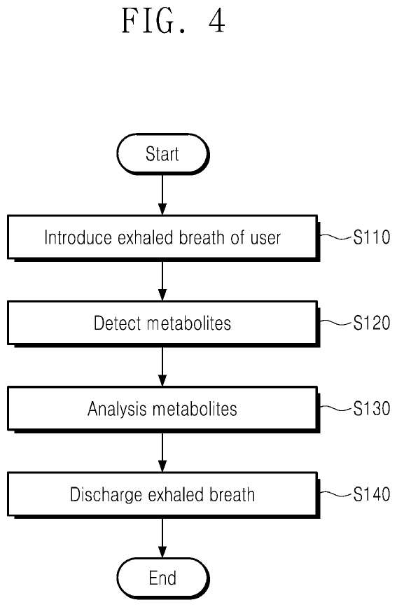

FIG. 4 is a flowchart illustrating an operating method of the apparatus for measuring fat oxidation 10 shown in FIGS. 1 and 2. The operating method of the apparatus for measuring fat oxidation 10 may include steps S110 to S140.

Referring to FIG. 4, in step S110, a user may introduce exhaled breath into the apparatus for measuring fat oxidation 10 through the sample injection unit 100. In step S120, the breath measurement unit 200 may detect metabolites in the exhaled breath. In step S130, the signal analysis unit 300 may analyze metabolites in the exhaled breath. In step S140, the exhaust unit 500 may discharge the exhaled breath in the apparatus for measuring fat oxidation 10 to the outside.

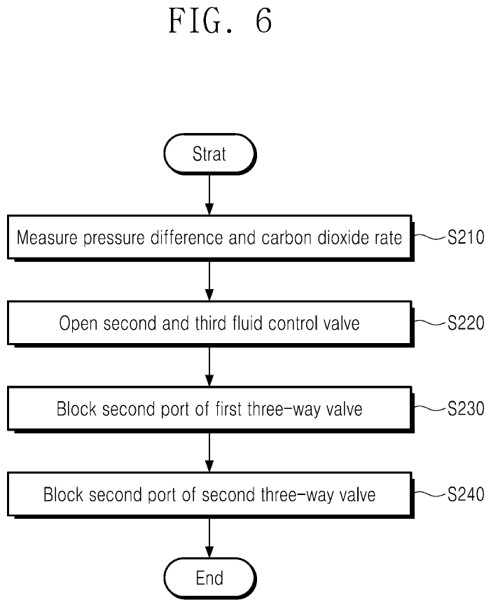

In one embodiment, the flow control unit 400 may control a volume of exhaled breath during steps S110 to S140. An operating method of the flow control unit 400 will be described later with reference to FIGS. 6 to 11.

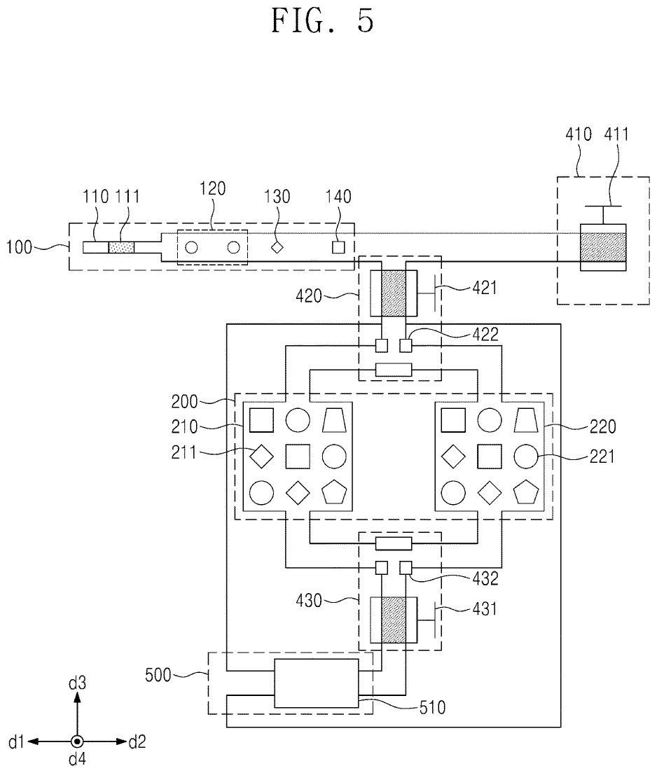

FIG. 5 illustrates a state in which the apparatus for measuring fat oxidation 10 is in a standby state. When the apparatus for measuring fat oxidation 10 is in the standby state, the flow control unit 400 may block the first to third fluid control valves 411, 421, and 431. When the apparatus for measuring fat oxidation 10 is in the standby state, the flow control unit 400 may open both the first three-way valve 422 and the second three-way valve 432. When the apparatus for measuring fat oxidation 10 is in the standby state, gas may pass between the first gas chamber 210 and the second gas chamber 220.

In one embodiment, the first three-way valve 422 and the second three-way valve 432 may each have a first port, a second port, and a third port corresponding to a first direction d1, a second direction d2, and a third direction d3. When the first port of the first three-way valve 422 and the first port of the second three-way valve 432 are opened, exhaled breath may be introduced into the first gas chamber 210. When the second port of the first three-way valve 422 and the second port of the second three-way valve 432 are opened, exhaled breath may be introduced into the second gas chamber 220.

FIG. 6 is a flowchart illustrating an operating method of the apparatus for measuring fat oxidation 10 when oral breath gas is introduced. The operating method may include steps S210 to S240.

Referring to FIG. 6, in step S210, the differential pressure sensor 120 may measure a pressure difference of the introduced exhaled breath. In step S210, the carbon dioxide sensor 140 may measure a carbon dioxide ratio of the exhaled breath. In step S220, the flow control unit 400 may open the second and third fluid control valves 421 and 431. In step S230, the flow control unit 400 may block the second port located in the second direction d2 of the first three-way valve 422. In step S240, the flow control unit 400 may block the second port located in the second direction d2 of the second three-way valve 432.

In one embodiment, when a pressure difference equal to or less than a predetermined value is measured by the differential pressure sensor 120 and a predetermined ratio of carbon dioxide is measured by the carbon dioxide sensor 140, the flow control unit 400 may determine the exhaled breath as oral breath gas. Oral breath gas and alveolar breath gas may be distinguished based on a concentration of carbon dioxide in the exhaled breath. When the carbon dioxide concentration in the exhaled breath is less than a predetermined threshold value (e.g., 1%), the exhaled breath may be classified as oral breath gas.

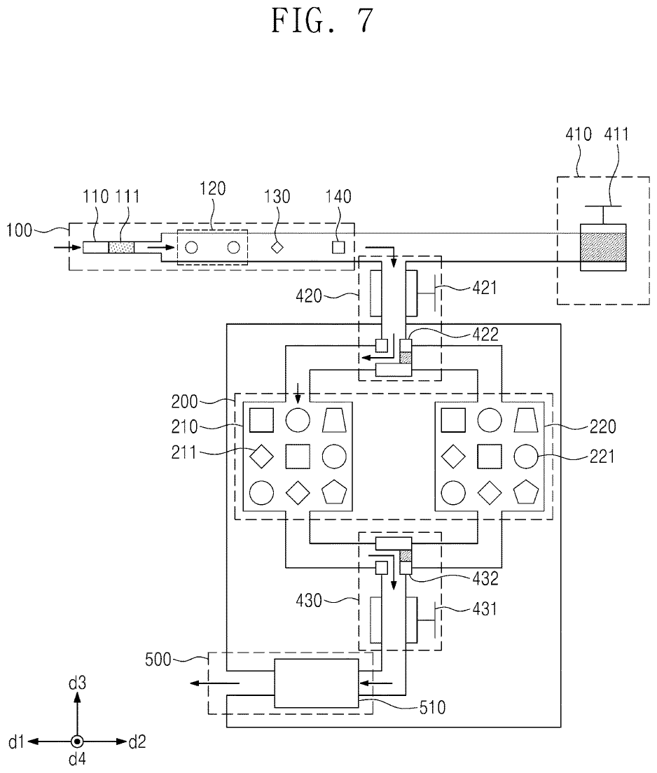

FIG. 7 illustrates a flow of oral breath gas in the apparatus for measuring fat oxidation 10. Referring to FIG. 7, the oral breath gas may be introduced through the sample injection unit 100. The oral breath gas may pass through the second fluid control valve 421 and then pass through the first port of the first three-way valve 422. The first gas chamber 210 may store the oral breath gas. When metabolite analysis of the oral breath gas is completed by the signal analysis unit 300, the oral breath gas may pass through the first port of the second three-way valve 432 and then pass through the third fluid control valve 431. The exhaust pump 510 may discharge the oral breath gas to the outside.

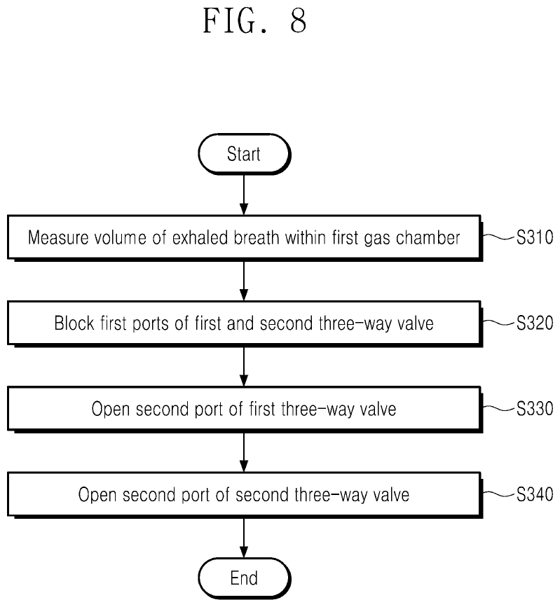

FIG. 8 is a flowchart illustrating an operating method of the apparatus for measuring fat oxidation 10 when alveolar breath gas is introduced. The operating method may include steps S310 to S340.

Referring to FIG. 8, in step S310, the flow sensor 130 may measure a volume of introduced exhaled breath. In step S310, the carbon dioxide sensor 140 may measure a carbon dioxide ratio of the exhaled breath. In step S320, the flow control unit 400 may block the first ports located in the first direction d1 of the first three-way valve 422 and the second three-way valve 432, respectively. In step S330, the flow control unit 400 may open the second port located in the second direction d2 of the first three-way valve 422. In step S340, the flow control unit 400 may open the second port located in the second direction d2 of the second three-way valve 432.

In one embodiment, when the flow sensor 130 detects that a predetermined volume of exhaled breath has been introduced into the first gas chamber 210 and a predetermined ratio of carbon dioxide is measured by the carbon dioxide sensor 140, the flow control unit 400 may determine the exhaled breath as alveolar breath gas. Oral breath gas and alveolar breath gas may be distinguished based on a concentration of carbon dioxide in the exhaled breath. When the carbon dioxide concentration in the exhaled breath is equal to or greater than a predetermined threshold value (e.g., 3%), the exhaled breath may be classified as alveolar breath gas.

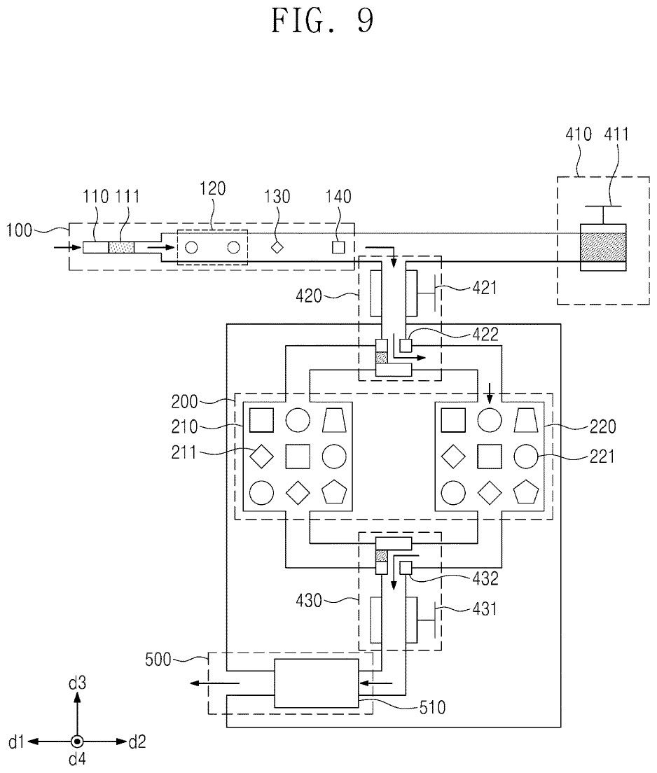

FIG. 9 illustrates a flow of alveolar breath gas in the apparatus for measuring fat oxidation 10. Referring to FIG. 9, the alveolar breath gas may be introduced through the sample injection unit 100. The alveolar breath gas may pass through the second fluid control valve 421 and then pass through the second port of the first three-way valve 422. The second gas chamber 220 may store the alveolar breath gas. When metabolite analysis of the alveolar breath gas is completed by the signal analysis unit 300, the alveolar breath gas may pass through the second port of the second three-way valve 432 and then pass through the third fluid control valve 431. The exhaust pump 510 may discharge the alveolar breath gas to the outside.

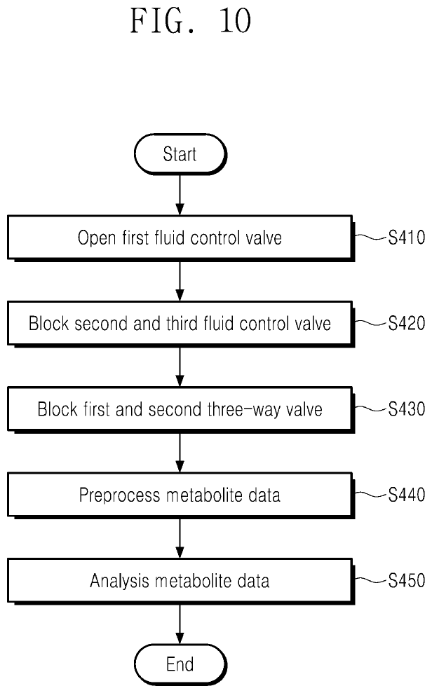

FIG. 10 is a flowchart illustrating an operating method for analyzing metabolites in the apparatus for measuring fat oxidation 10. The operating method may include steps S410 to S450.

Referring to FIG. 10, in step S410, the flow control unit 400 may open the first fluid control valve 411. In step S420, the flow control unit 400 may block the second and third fluid control valves 421 and 431. In step S430, the flow control unit 400 may block the second ports of the first and second three-way valves 422 and 432. In step S440, the preprocessing unit 310 may perform preprocessing on data detected by the first gas sensor array 211 and the second gas sensor array 221. In step S450, the calculation unit 320 may perform analysis on the preprocessed data.

In one embodiment, data detected by the first gas sensor array 211 and the second gas sensor array 221 may pass through the instrumentation amplifier circuit 311, the automatic amplification circuit 312, and the capacitive filter 313. The preprocessing unit 310 may be configured to maximize a SNR of the data based on the data passing through the instrumentation amplifier circuit 311. The preprocessing unit 310 may be configured to remove an offset of the data based on the data passing through the automatic amplification circuit 312. The preprocessing unit 310 may be configured to remove high-frequency noise and white noise of the data based on the data passing through the capacitive filter 313.

In one embodiment, the calculation unit 320 may normalize the preprocessed data and generate two-dimensional response map data based on the preprocessed data passing through the computing circuit 321. The calculation unit 320 may train the two-dimensional response map data through the evaluation circuit 322. The calculation unit 320 may extract feature points from the two-dimensional response map data and evaluate a concentration and composition of metabolites in exhaled breath.

In one embodiment, the evaluation circuit 322 of the calculation unit 320 may operate based on an AI algorithm. The evaluation circuit 322 may use a learning algorithm based on a 1D-CNN suitable for normalized time-series data.

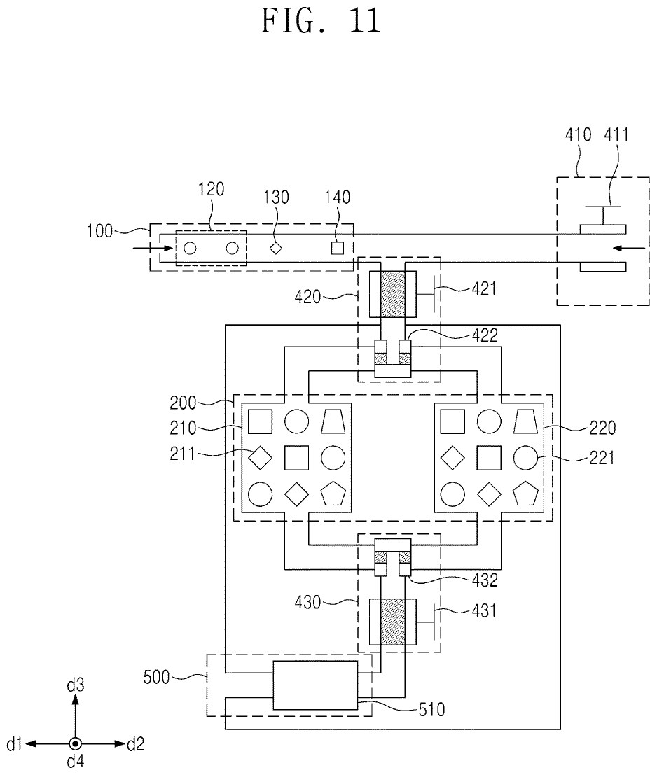

FIG. 11 illustrates a flow of exhaled breath during metabolite analysis in the apparatus for measuring fat oxidation 10. Referring to FIG. 11, based on the start of operation of the signal analysis unit 300, the flow control unit 400 may open the first fluid control valve 411. The flow control unit 400 may block the second and third fluid control valves 421 and 431. The flow control unit 400 may block the first and second three-way valves 422 and 432.

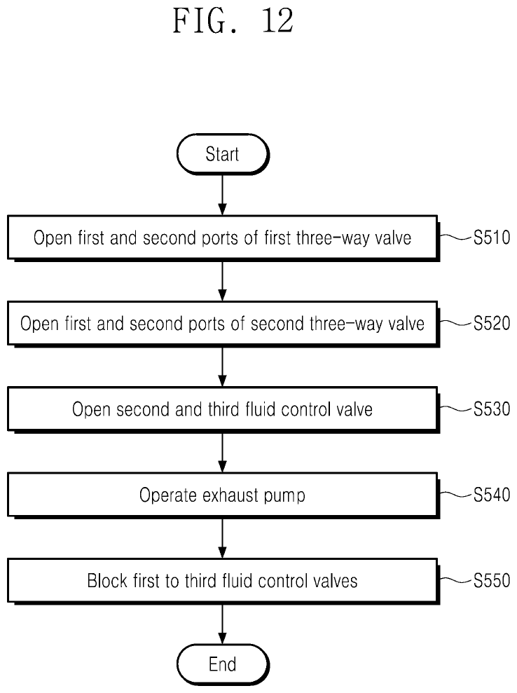

FIG. 12 is a flowchart illustrating an operating method for discharging exhaled breath in the apparatus for measuring fat oxidation 10. The operating method may include steps S510 to S550.

Referring to FIG. 12, in step S510, the flow control unit 400 may open the first port and the second port of the first three-way valve 422. In step S520, the flow control unit 400 may open the first port and the second port of the second three-way valve 432. In step S530, the flow control unit 400 may open the second and third fluid control valves 421 and 431. In step S540, the exhaust unit 500 may start operation of the exhaust pump 510. In step S550, the flow control unit 400 may block the first to third fluid control valves 411, 421, and 431.

In one embodiment, based on the opening of the first port and the second port of the first three-way valve 422, the first gas chamber 210 and the second gas chamber 220 may be connected to the sample injection unit 100. Based on the opening of the first port and the second port of the second three-way valve 432, the first gas chamber 210 and the second gas chamber 220 may be connected to the exhaust unit 500.

In one embodiment, based on the opening of the second and third fluid control valves 421 and 431, the exhaust unit 500 may start operation of the exhaust pump 510. The exhaust pump 510 may introduce external air through the sample injection unit 100. The exhaust pump 510 may discharge residual exhaled breath remaining in the apparatus for measuring fat oxidation 10 to the outside. The exhaust pump 510 may operate for a predetermined period of time.

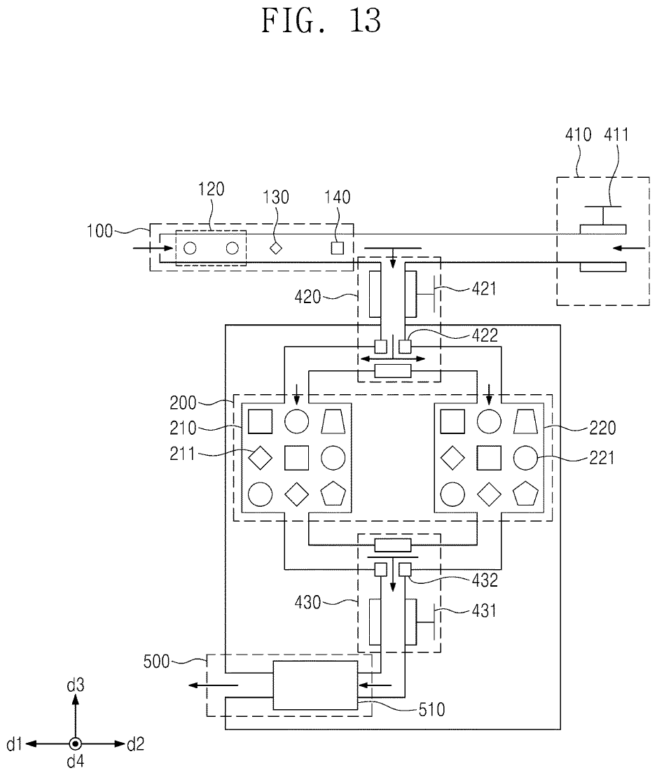

FIG. 13 illustrates flows of external air introduced from the outside and residual exhaled breath discharged to the outside in the apparatus for measuring fat oxidation 10.

Referring to FIG. 13, when the second and third fluid control valves 421 and 431 are opened, external air may be introduced through the sample injection unit 100 based on operation of the exhaust pump 510. When the external air passes through the second fluid control valve 421 and the first port and the second port of the first three-way valve 422 and is introduced into the first gas chamber 210 and the second gas chamber 220, residual exhaled breath in the first and second gas chambers 210 and 220 may be discharged to the outside through the first port and the second port of the second three-way valve 432, the third fluid control valve 431, and an outlet. The external air may also be discharged to the outside through the first port and the second port of the second three-way valve 432, the third fluid control valve 431, and the outlet. Based on the discharge of the external air, the first to third fluid control valves 411, 421, and 431 may be blocked. The apparatus for measuring fat oxidation 10 may return to the same state as the apparatus for measuring fat oxidation 10 shown in FIG. 5.

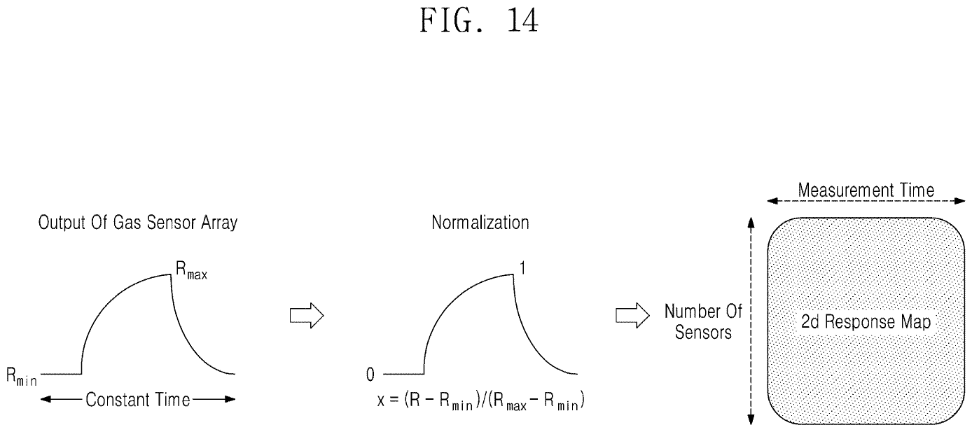

FIG. 14 is an exemplary diagram illustrating a data normalization process and a two-dimensional data map generation process in the computing circuit 321.

Referring to FIG. 14, data measured by the first and second gas sensor arrays 211 and 221 may be output data based on changes in electrical characteristics, such as current, resistance, and voltage. The computing circuit 321 may normalize data measured by the sensors into data between 0 and 1 based on a minimum value and a maximum value of the measured data. The computing circuit 321 may generate two-dimensional response map data based on the normalized data.

In one embodiment, when a maximum value of data measured from the gas sensor array is Rmax and a minimum value thereof is Rmin, data R may be normalized according to the following equation. In Equation 1, x represents normalized data (0≤x≤1).

x=(R−Rₘᵢₙ)/(Rₘₐₓ−Rₘᵢₙ) Equation 1

In one embodiment, the two-dimensional response map data may be configured in a matrix form of [number of gas sensors×measurement time].

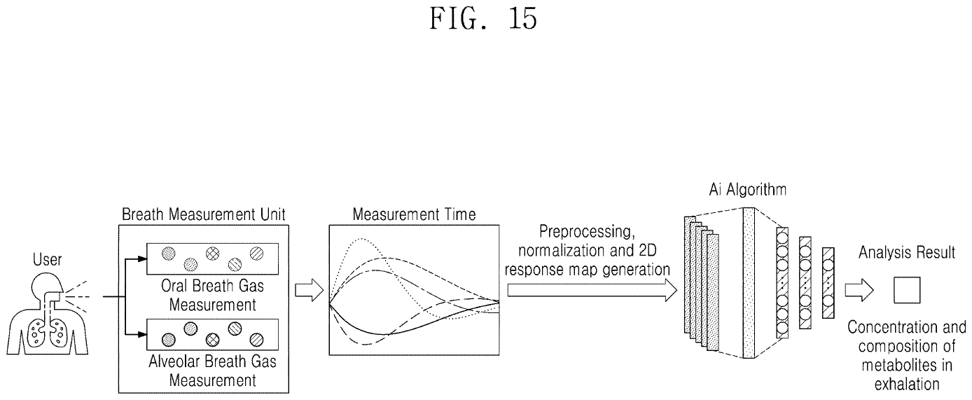

FIG. 15 is a schematic diagram illustrating a process of analyzing metabolites in exhaled breath using an AI algorithm of the evaluation circuit 322.

Referring to FIG. 15, a user may introduce exhaled breath through the sample injection unit 100. Sensors included in the sample injection unit 100 may classify the exhaled breath of the user into oral breath gas and alveolar breath gas. The breath measurement unit 200 may detect metabolites in exhaled breath for each of the oral breath gas and the alveolar breath gas. The preprocessing unit 310 of the signal analysis unit 300 may remove signal noise and offset from metabolite measurement result data of the exhaled breath. The calculation unit 320 of the signal analysis unit 300 may normalize the data and generate two-dimensional response map data. The calculation unit 320 may derive analysis results including a concentration and composition of metabolites from the two-dimensional response map data by using an AI algorithm based on a 1D-CNN.

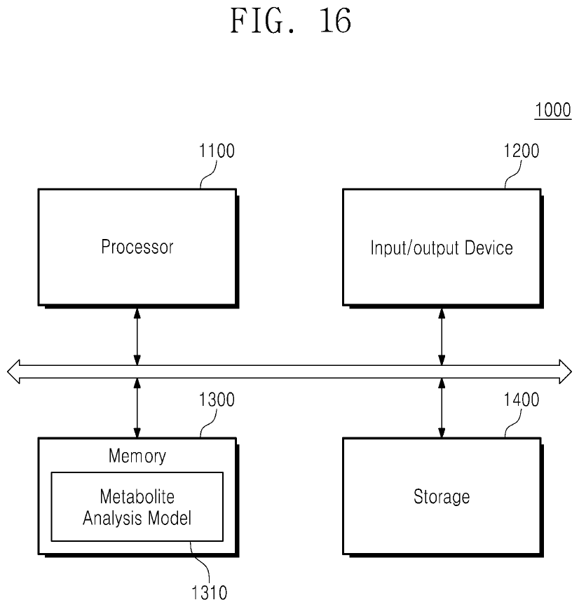

FIG. 16 illustrates a block diagram of an electronic device according to embodiments of the present disclosure. Referring to FIG. 16, an electronic device 1000 may include a processor 1100, an input/output device 1200, a memory 1300, and a storage 1400. The electronic device 1000 may represent the apparatus for measuring fat oxidation 10 described in the present disclosure (described with reference to FIGS. 1 and 2).

The processor 1100 may perform functions as a central processing unit of the electronic device 1000. The processor 1100 may perform control operations and calculation operations required for data management and result generation of the electronic device 1000. For example, under control of the processor 1100, the apparatus for measuring fat oxidation 10 may collect and analyze metabolite data in exhaled breath. The preprocessing unit 310 and the calculation unit 320 included in the signal analysis unit 300 of FIG. 2 may be configured as separate hardware devices that assist or replace metabolite data collection and analysis of the processor 1100. The processor 1100 may operate by utilizing a computing space of the memory 1300 and may read files for driving an operating system from the storage 1400. The processor 1100 may execute an operating system and various applications. In another example, under control of the processor 1100, the input/output device 1200 may receive target metabolites from a user and may provide metabolite analysis results to the user.

The input/output device 1200 may include an interface or a developer interface for communicating with a user of the electronic device 1000. For example, the input/output device 1200 may include at least one of various output devices, such as a monitor, a printer, or a lamp. The input/output device 1200 may include at least one of various input devices, such as a keyboard, a touch pad, a mouse, and a microphone.

The memory 1300 may store data and process codes processed or to be processed by the processor 1100. For example, the memory 1300 may store information for driving various sensors included in the sample injection unit 100, information related to gas sensor arrays included in the breath measurement unit 200, information related to target metabolites, and information for preprocessing and analyzing data. The memory 1300 may be used as a main memory of the electronic device 1000. The memory 1300 may include DRAM (Dynamic RAM), SRAM (Static RAM), PRAM (Phase-change RAM), MRAM (Magnetic RAM), FeRAM (Ferroelectric RAM), RRAM (Resistive RAM), and the like.

A metabolite analysis model 1310 may be loaded into and executed in the memory 1300. The metabolite analysis model 1310 may implement the AI algorithm used in the evaluation circuit 322 included in the calculation unit 320 of the signal analysis unit 300 shown in FIG. 3. The metabolite analysis model 1310 may be a part of a computing space of the memory 1300. For example, firmware may be stored in the storage 1400 and may be loaded into the memory 1300 when executed. The processor 1100 may execute the firmware loaded into the memory 1300.

The storage 1400 may store data generated for long-term storage purposes by an operating system or applications, files for driving an operating system, or executable files of applications. For example, the storage 1400 may store files for executing the metabolite analysis model 1310. The storage 1400 may be used as an auxiliary memory of the electronic device 1000. The storage 1400 may include flash memory, PRAM, MRAM, FeRAM, RRAM, and the like.

Although embodiments of the present disclosure have been described in detail above, the scope of the present disclosure is not limited thereto, and various modifications and improvements made by those skilled in the art using the basic concept of the present disclosure also fall within the scope of the present disclosure.

An apparatus for measuring fat oxidation according to the present invention can easily and quickly check the state of fat oxidation state in the user's body by measuring the user's oral breath gas and alveolar breath gas separately and comparing and analyzing the metabolites in the two breath gases. And the apparatus can contribute to the promotion of national health and reduction of medical expenses by monitoring the user's diet status in real time. Also, the apparatus can be utilized in various fields such as early screening tests based on exhalation of respiratory diseases.

What is claimed is:

1. An apparatus for measuring fat oxidation, comprising:
a sample injection unit into which exhaled breath of a user is introduced;
a breath measurement unit configured to detect metabolites in the exhaled breath;
a signal analysis unit configured to analyze the detected metabolites;
a flow control unit configured to automatically control a flow rate of the exhaled breath; and
an exhaust unit configured to discharge the exhaled breath to the outside.

2. The apparatus of claim 1,
wherein the sample injection unit comprises:
a straw configured to directly contact an oral cavity of the user;
a differential pressure sensor configured to detect introduction of the exhaled breath into the breath measurement unit;
a flow sensor configured to control a volume of the exhaled breath introduced into the breath measurement unit; and
a carbon dioxide sensor configured to classify the exhaled breath into oral breath gas and alveolar breath gas.

3. The apparatus of claim 2,
wherein the straw further comprises a droplet filter configured to block droplets.

4. The apparatus of claim 1,
wherein the breath measurement unit comprises:
a first gas chamber configured to store oral breath gas included in the exhaled breath; and
a second gas chamber configured to store alveolar breath gas included in the exhaled breath.

5. The apparatus of claim 4,
wherein the first gas chamber comprises a first gas sensor array configured to detect metabolites in the exhaled breath, and
the second gas chamber comprises a second gas sensor array configured to detect metabolites in the exhaled breath.

6. The apparatus of claim 5,
wherein the first gas sensor array comprises at least one of a semiconductor gas sensor, an electrochemical gas sensor, a photoionization gas sensor, a metal organic framework (MOF) gas sensor, a carbon nanowall (CNW) gas sensor, or a bioreceptor gas sensor, and
the second gas sensor array comprises the same gas sensors as the first gas sensor array.

7. The apparatus of claim 1,
wherein the signal analysis unit comprises:
a preprocessing unit configured to preprocess metabolite data signals detected by the breath measurement unit; and
a calculation unit configured to calculate and analyze a concentration and composition of metabolites included in the exhaled breath based on the preprocessed signals.

8. The apparatus of claim 7,
wherein the preprocessing unit comprises:
an instrumentation amplifier circuit configured to maximize a signal-to-noise ratio (SNR) of the metabolite data signals measured by the breath measurement unit;
an automatic amplification circuit configured to remove an offset from the measured signals; and
a capacitive filter configured to remove high-frequency noise and white noise from the measured signals.

9. The apparatus of claim 7,
wherein the calculation unit comprises:
a computing circuit configured to normalize data based on the preprocessed signals and generate two-dimensional response map data; and
an evaluation circuit configured to analyze results output from the computing circuit.

10. The apparatus of claim 1,
wherein the flow control unit comprises:
a first flow control unit connected to the sample injection unit;
a second flow control unit connected between the sample injection unit and the breath measurement unit; and
a third flow control unit connected between the breath measurement unit and the exhaust unit.

11. The apparatus of claim 10,
wherein the first flow control unit comprises a first fluid control valve,
the second flow control unit comprises a second fluid control valve and a first three-way valve, and
the third flow control unit comprises a third fluid control valve and a second three-way valve.

12. The apparatus of claim 1,
wherein the exhaust unit further comprises an exhaust pump configured to discharge residual exhaled breath to the outside.

13. A method of operating an apparatus for measuring fat oxidation by measuring metabolites in exhaled breath to determine a fat oxidation state in a body, the method comprising:
introducing exhaled breath of a user into the apparatus by a sample injection unit included in the apparatus;
detecting metabolites included in the exhaled breath by a breath measurement unit included in the apparatus;
analyzing the metabolites by a signal analysis unit included in the apparatus; and
discharging the exhaled breath to the outside by an exhaust unit included in the apparatus.

14. The method of claim 13,
wherein the method further comprises controlling a flow rate of the exhaled breath in real time by a flow control unit included in the apparatus.

15. The method of claim 13,
wherein introducing the exhaled breath into the apparatus comprises:
measuring a pressure difference and a carbon dioxide ratio of the exhaled breath by a differential pressure sensor and a carbon dioxide sensor included in the sample injection unit;
opening a second fluid control valve and a third fluid control valve by a flow control unit included in the apparatus;
blocking second ports of a first three-way valve and a second three-way valve by the flow control unit;
introducing the exhaled breath into a first gas chamber of the breath measurement unit by the flow control unit; and
operating an exhaust pump included in the exhaust unit by the exhaust unit.

16. The method of claim 15,
wherein introducing the exhaled breath into the apparatus further comprises:
measuring a volume of exhaled breath within the first gas chamber by a flow sensor included in the sample injection unit;
measuring a carbon dioxide ratio of the exhaled breath by the carbon dioxide sensor included in the sample injection unit;
blocking first ports of the first three-way valve and the second three-way valve by the flow control unit;
opening second ports of the first three-way valve and the second three-way valve by the flow control unit;
introducing the exhaled breath into a second gas chamber of the breath measurement unit by the flow control unit; and
operating the exhaust pump included in the exhaust unit by the exhaust unit.

17. The method of claim 13,
wherein detecting metabolites included in the exhaled breath comprises:
measuring volumes of exhaled breath introduced into a first gas chamber and a second gas chamber of the breath measurement unit by a flow sensor included in the sample injection unit;
opening a first fluid control valve and blocking a second fluid control valve and a third fluid control valve by a flow control unit included in the apparatus;
blocking second ports of a first three-way valve and a second three-way valve by the flow control unit; and
measuring metabolites included in the exhaled breath by gas sensor arrays included in the breath measurement unit.

18. The method of claim 13,
wherein analyzing the metabolites comprises:
preprocessing metabolite data by a preprocessing unit included in the signal analysis unit;
normalizing the preprocessed data and generating two-dimensional response map data based on the normalized data by a calculation unit included in the signal analysis unit; and
analyzing a concentration and composition of metabolites included in the exhaled breath by the calculation unit.

19. The method of claim 13,
wherein discharging the exhaled breath to the outside comprises:
opening a first port and a second port of a first three-way valve by a flow control unit included in the apparatus;
opening a first port and a second port of a second three-way valve by the flow control unit;
opening a second fluid control valve and a third fluid control valve by the flow control unit; and
operating an exhaust pump by the exhaust unit.

20. The method of claim 13,
wherein discharging the exhaled breath to the outside further comprises blocking first to third fluid control valves included in the apparatus by a flow control unit.
